(19) United States
(12) Patent Application Publication
(10) Pub. No.: US 2008/0269328 A1
(43) Pub. Date: Oct. 30, 2008

(54) Effects of low n-6:n-3 fatty acid ratio from two dietary sources on plasma and tissue lipid composition

(76) Inventor: Mohammed Moghadasian, Winnipeg (CA)

Correspondence Address: ADE & COMPANY INC., 2157 Henderson Highway, WINNIPEG, MB, R2G1P9, CA

(21) Appl. No.: 12/100,076

(22) Filed: Apr. 9, 2008

Related U.S. Application Data
(60) Provisional application No. 60/910,707, filed on Apr. 9, 2007.

(51) Int. Cl. A61K 31/19; A23D 9/00

(52) U.S. Cl. 514/557; 426/601

(57) ABSTRACT

Health benefits from low n-6:n-3 fatty acid (FA) ratio on cardiovascular risk have been shown. However, the impact of the source of n-3 FAs has not been fully investigated. Our purpose was to investigate cardiovascular benefits of oils with a low ratio of n-6:n-3 FAs, but different sources of n-3 FAs. Mice were divided into 3 groups (n=7) and fed a diet supplemented with either a fish or flaxseed oil-based ‘designer oils’ with an approximate n-6:n-3 FA ratio of 2/1 or with a safflower-oil-based diet with a ratio of 25/1, for 16 weeks. Plasma lipids and fatty acid profile of the liver and heart tissues were characterized. Plasma cholesterol levels were reduced in both fish and flax groups by 27% and 36%, respectively, as compared to controls at endpoint. The levels of EPA and DHA in liver phospholipids were significantly increased in both fish and flax groups as compared to the control group, with more profound increases in the fish group. Arachidonic acid levels were similarly decreased in the liver tissues from both fish and flax groups as compared to controls. Our data indicates that health benefits may be achieved by lowering dietary n-6:n-3 FA even in a high fat diet medium.

### PRIOR APPLICATION INFORMATION

This application claims the benefit of U.S. Provisional Patent Application 60/910,707, filed Apr. 9, 2007.

### BACKGROUND OF THE INVENTION

Cardiovascular disease still remains as one of the leading causes of disability and mortality. Epidemiological and clinical studies have reported a beneficial effect of both a-linolenic acid (ALA, 18:3n-3) and eicosapentaenoic acid (EPA, 20:5n-3)/docosahexaenoic acid (DHA, 22:6n-3) on the cardiovascular system [23,12] through improvements in blood lipid profile, by improving lipoprotein profiles [27,7], decreasing blood pressure [18], and reducing inflammatory proteins, (IL-1β, IL-6, TNF-α, IL-12, and interferon-6) [14, 16, 25, 43]. The largest dietary source of ALA is flaxseed oil, while marine products are the main natural sources of EPA/DHA. Majority of research to date has focused on EPA/DHA on cardiovascular health, while limited studies on ALA benefits are available. Also, the majority of these studies have involved supplementation of n-3 fatty acids, without fully controlling for background diet.

ALA can partially be converted to EPA/DHA endogenously. However, this conversion is not very efficient in animals and humans. Furthermore, it is suggested that the extent of this conversion can be species- and tissue-specific [6, 1, 5]. n-6 (arachidonic acid) and n-3 (EPA) fatty acids also compete for the cyclooxygenase (COX) and lipoxygenase (LOX) enzymes necessary for the formation of eicosanoids [36]. Pro-inflammatory and pro-aggregatory series 2 eicosanoids are derived from arachidonic acid (ARA, 20:4n-6), whereas less inflammatory and less aggregatory series 3 eicosanoids, namely prostaglandin, thromboxane and 5 series of leukotrienes, are produced from EPA [37, 17]. The ratio of n-6:n-3 fatty acids is, therefore, hypothesized to be important for production of optimal effects on human health. For that reason, a n-6:n-3 fatty acid ratio of 2-4:1 has recently been suggested for the general population [36].

To our knowledge, the impact of a high fat diet low in n-6 to n-3 fatty acid ratio on several cardiovascular risk factors in an established animal model has not been fully documented. Thus, the purpose of the present study was to investigate cardiovascular benefits of diets supplemented with “designer oils” containing low ratios of n-6:n-3 fatty acids from different sources, either fish oil (source of DHA and EPA) or flaxseed oil (source of ALA), in C57BL/6 mice fed a high-fat diet. It must be mentioned that previous studies investigated the effects of supplementation with either fish oil or flaxseed oil on plasma lipid profile. However, the current study deals with new types of oils which are composed of similar amounts of saturated and polyunsaturated fatty acids and low ratio of n-6:n-3 FAs but from different sources of n-3 FAs. Therefore, this study may mimic the nature of a free living lifestyle with relatively high dietary fat intake and aims to investigate the effects of low n-6:n-3 FAs ratio in such a condition in a wild-type animal commonly used for studying human diseases.

### SUMMARY OF THE INVENTION

According to a first aspect of the invention, there is provided an edible oil having an n-6:n-3 ratio of approximately 1.5:1 to 4:1.

The edible oil may have an n-6:n-3 ratio of approximately 2:1 to 3:1.

The edible oil may have a fatty acid profile of approximately 30-40% n-6 fatty acid and 10-25% n-3 fatty acid.

The edible oil may have a fatty acid profile of approximately 33-38% n-6 and 15-20% n-3.

According to a second aspect of the invention, there is provided a method of improving over-all health comprising administering to an individual in need of such treatment an effective amount of an edible oil having an n-6:n-3 ratio of approximately 1.5:1 to 4:1.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Health benefits from low n-6:n-3 fatty acid (FA) ratio on cardiovascular risk have been shown. However, the impact of the source of n-3 FAs has not been fully investigated. Our purpose was to investigate cardiovascular benefits of oils with a low ratio of n-6:n-3 FAs, but different sources of n-3 FAs in C57BL/6 mice. As discussed below, twenty-one mice were divided into 3 groups (n=7) and fed a diet supplemented with either a fish or flaxseed oil-based ‘designer oils’ with an approximate n-6:n-3 FA ratio of 2/1 or with a safflower-oil-based diet with a ratio of 25/1, for 16 weeks. Plasma lipids and fatty acid profile of the liver and heart tissues were characterized. As discussed below, compared to baseline, plasma triacylglycerol levels declined (>50%) in all groups by week 4. Plasma cholesterol levels were reduced in both fish and flax groups by 27% and 36%, respectively, as compared to controls at endpoint. The levels of EPA and DHA in liver phospholipids were significantly increased in both fish and flax groups as compared to the control group, with more profound increases in the fish group. Arachidonic acid levels were similarly decreased in the liver tissues from both fish and flax groups as compared to controls. Our data indicates that health benefits may be achieved by lowering dietary n-6:n-3 FA even in a high fat diet medium.

This study has characterized the effects of low dietary ratios of n-6:n-3 fatty acids, from different sources, on plasma lipids, liver lipids and liver fatty acid profiles in wild-type mice. “Designer oils” with a low n-6:n-3 ratio as a part of a high fat diet displayed beneficial effects on cardiovascular risk factors and tissue composition in C57BL/6 mice, regardless of the origin of n-3 fatty acids. Improvements in blood lipids (TAG, TC, and non-HDL cholesterol) were comparable between the flax and fish groups. However, the extent of increases in DHA levels in liver tissues was greater in the fish group as compared to either flax or control groups.

These “designer oils” are carefully made to contain both essential and non-essential fats for normal human body function, while at the same time are capable of promoting human health and quality of life. Essential fats are those which the human body cannot make, but need them for normal body structure and function, and therefore, these fats must come from diets. Non-essential fats are those that the body needs them for energy, and normal structure and function, but our body can make them. It is generally recommended that humans consume various types of fats including essential and non-essential. However, the proportion of these fats in the diet seems to be very influential on human health. Therefore, these “designer oils” purposely contain appropriate amounts of essential and non-essential fats. In this regard, these oils are unique as literature search does not show any such types of oil. In previous research, the impact of some essential fats including namely n-3 and n-6 fatty acids has been separately investigated on health parameters. However, unlike those studies, our “designer oils” provide a naturally acceptable combination of various fats with an end point of providing appropriate amounts of these fats for energy production, body structure purposes and over-all optimal body function. Several lines of research have demonstrated that nutrients including fats can interact with each other in human body to produce an effect on body function. In this regard, our oils have the right amounts of each class of fats to allow an appropriate interaction for normal body function, and such conditions have not been previously investigated. Furthermore, the carefully incorporated various types of fats make these “designer oils” health-promoting. We have already observed evidence for reductions in common cardiovascular risk factors in mice including but by no means limited to improvements in blood lipid profile, body fat accumulation, liver abnormalities, beneficial alterations in tissue (including the heart) fat composition, beneficial alterations in mediators involved in regulation of immune system and inflammatory pathways. In addition to these biological benefits, our oils will contain adequate amounts of natural lipid-soluble vitamins including vitamin E, will be more resistant to auto-oxidation and therefore have longer shelf life (as compared to for example flaxseed oil), will have very good taste and flavor (for example unlike fish oil (fishy taste), or sunflower oil (grassy taste)).

These oils can be used in home kitchens, restaurants, fast food establishments, food making facilities, margarine, salad dressing, and bakery. This wide variety of applications of “designer oils” indicates that consumers are given opportunities to incorporate high quality fat in their daily diet without being concerned of over-using a harmful type of fat. At the same time, consumption of these oils will remove the need for supplementation with either flaxseed oil or fish oil, a practice that is currently highly recommended but produces very little consumer compliance due to the inconvenience of swallowing of capsules, unacceptable taste for some individuals, after-use problems such as bad burping and stomach disturbance, environment friendly, and more importantly cost-effectiveness and convenience of use as our oils will be part of individuals' food rather than taking a separate supplement formulation.

In a preferred embodiment, as shown in Table 1, the designer oil comprises an edible oil having an n-6:n-3 ratio of approximately 1.5:1 to 4:1 or more preferably from 2:1 to 3:1. More specifically, the designer oil comprises a fatty acid profile wherein the ratio of 18:2 (n-6) to 18:3 (n-3)+20:5 (n-3)+22:6 (n-3) is approximately 1.5:1 to 4:1 or more preferably from 2:1 to 3:1. More specifically, the designer oil may be 30-40% n-6 fatty acid, for example, 18:2, and 10-25% n-3 fatty acid, for example, 18:3 (n-3), 20:5 (n-3) and 22:6 (n-3). More preferably, the edible designer oil may be 33-38% n-6 and 15-20% n-3.

Diets supplemented with the “designer oils” were apparently well tolerated. The animals gained weight at a steady and comparable rate during the experimental course. This was similar to our previous observation that addition of fish oil or flaxseed into the diet of apo E-KO mice or rabbits, respectively, did not alter body weight gain as compared to corresponding control groups [40, 13]. However, Barcelli et al. [4] reported that rats fed either a high n-6 (from safflower oil) or high n-3 (from fish oil) diet gained more weight than rats fed a high saturated fat diet, while all diets contained identical amounts of total fat. These observations suggest the importance of the quality of dietary fat on body weight gain independent of their energy impact.

Although body weight was comparable among groups in the present study, food intake was not. While not wishing to be bound to a particular hypothesis, it is possible that either the absorption of dietary fat was impaired and, therefore, unabsorbed fat was excreted, or the interactions between high levels of n-3 fatty acids and other fatty acids may prevent additional body weight gain. One may also speculate that these formulations may induce β-oxidation system, preventing fat accumulation and body weight gain.

Plasma TAG-lowering properties of n-3 fatty acids were observed in both flax and fish groups as early as 4 weeks following the treatment; this is in agreement with previous observations [22, 20]. It was interesting to note that the control diet also reduced plasma TAG levels in this model. The reductions in plasma TAG levels in the control group may be related to presence of relatively high levels (>22%) of total monounsaturated fatty acids in the control diet. Another speculation for such an apparent paradox of the control diet on plasma TAG levels may be made with regard to potential species-related variations or interactions between dietary oils and carbohydrate. Regardless, it remains to be answered whether the significant reductions observed in plasma TAG levels in the control group or in the treated groups were mediated through the same mechanisms. It is likely that the “control oil formulation” acted through distinct mechanisms from those of the “n-3 fatty acid formulations.”

In the present study, both treatment groups significantly reduced plasma TC and non-HDL cholesterol levels as compared to controls. These observations provide evidence for the cholesterol-lowering effects of dietary n-3 fatty acids following consumption of a high fat diet. It remains to be answered whether these ‘designer oils’ could inhibit dietary cholesterol absorption or increase cholesterol catabolism and biliary excretion. Recently, a low dietary n-6:n-3 ratio, with EPA and DHA was also shown to reduce TC in LDL-deficient mice [39]. In addition, Du et al. [9] also reported reduced serum cholesterol in C57BL/6 mice fed either perilla oil (high ALA) or a high DHA/Soy diet compared to mice fed a diet enriched with safflower oil after 71 weeks. The study of Du et al. [9] suggested a decrease in HMG-CoA reductase activity in the fish and flax groups; this mechanism may also apply to our current study. On the other hand, other studies reported an increase in LDL-cholesterol concentrations in hypertriglyceridemic participants after treatment with DHA enriched eggs [27]. n-3 fatty acids have also been shown to favorably modify LDL particle size [32]. Our data showed a slight increase in HDL-cholesterol levels in the control group as compared to either of treated group at week 4; this reflects higher TC levels in the control group. On the other hand, by the end of the study the fish group showed higher HDL cholesterol levels; such an effect of fish oil has been also sporadically observed in other studies [7]. It is important to note the specialties in mouse lipoprotein metabolism; HDL is in the predominant lipoprotein in mice, unlike humans. Also, the CS7BL/6 strain presents with its own differences in lipoprotein metabolism [24].

Previous studies have shown the effects of low n-6:n-3 FA ratios from a single source of n-3 FAs on plasma lipids. For example, Yamashita et al. [41] reported significant reductions in plasma TAG and LDL and elevated HDL from dietary ALA as a part of a low n-6:n-3 ratio in mice.

Overall, both treatment diets substantially influenced liver lipid composition as compared to the controls. Yamazaki et al. [42] found that safflower oil induced fatty liver in C57BL/6J mice, but fish oil further exacerbated this negative effect. In contrast with Yamazaki and co-workers' observations that although we found high amounts of lipids in the liver of the control group, both treated groups displayed a significantly lower amount of total lipid. Yamazaki et al. [42] also observed a significant increase in hepatic Acetyl CoA oxidase activity as a result of safflower feeding. Our results are in agreement with findings from another study in which fish oil was shown to prevent fatty liver induced by perfluorooctanoic acid in mice [26] and reverse the condition [2]. Furthermore, both treatment diets resulted in lower n-6:n-3 FA ratios in various lipid fractions in the liver. Lower n-6:n-3 ratios and higher EPA/DHA concentrations in the liver of the flax groups (compared to control) may suggest a conversion of ALA to EPA/DHA. The literature has shown that ALA can be partially converted to EPA/DHA endogenously [21]; however, the efficiency is often questioned. Our data suggest that dietary ALA cannot be incorporated into the tissue phospholipids at an identical rate to that of dietary EPA/DHA. This important finding suggests that perhaps high levels of ALA may be able to induce hepatic desaturase and elongase enzymes necessary for conversion of ALA to EPA and DHA. It is important to note that this finding may be species- and/or tissue-related, as previously reported in rats [5]. Future studies are needed to properly address these issues. Nevertheless, dietary ALA significantly reduced tissue ARA in most lipid fractions to a level comparable to that achieved by dietary fish oil (EPA/DHA). These results are consistent with previous findings [19]. Tissue ARA may be as important as total tissue DHA because of its role in production of pro-inflammatory proteins. The effects of these ‘designer oils’ on inflammatory markers are a future area of investigation.

The incorporation of these fatty acids in liver cell membrane imply a similar profile in cardiac cell membrane, which aid in rationalizing the anti-arrhythmic effects of flaxseed oils [3] and fish oils [8]. Many animal studies [10, 28-30] have suggested that EPA and DHA have direct protective effects on the heart.

The reduction in liver total lipid may indicate beneficial cardiovascular implications as well. Low n-6:n-3 FA ratio may affect several metabolic pathways in the liver, including involvement of the peroxisome proliferator-activated receptors (PPARs), which consist of several sub-types and possess various functions related to lipid metabolism. This includes modification of fatty acid metabolism by reducing TAG synthesis/storage and increasing fatty acid oxidation [11].

Two new formulations of dietary oils containing a n-6:n-3 fatty acid ratio of approximately 2:1 as part of a high fat diet were tested in mice. Incorporation of n-3 fatty acids was observed in various lipid fractions tested. Although the levels of EPA and DHA in tissues from the fish oil-based treated group were statistically higher than those in the flaxseed oil-based treated animals, in some of the lipid fractions tested, the overall modifications in the tissue lipid profile seemed comparable. This may suggest that high amounts of dietary n-3 FAs, regardless of source, may produce beneficial alterations in tissue lipid profile. However, fish oil may be more potent in enhancing tissue DHA levels, but not in reducing the tissue ARA concentrations. However, the impact of n-3 FA-rich oil formulations on plasma TAG levels was comparable to that of n-6 FA-rich control oil.

### Results

### Body Weight and Food Intake

A steady increase in body weight in all groups of animals throughout the experimental course was observed. At baseline, the mean body weight was 26.3 g, 25.6 g, and 25.6 g, in the control, flax, and fish groups, respectively, the mean body weight in each group was respectively increased to 29.1 g, 29.3 g, and 29.7 g by week 8 and to 30.3 g, 31.3 g, and 31.5 g by the end of the study. Body weight did not differ significantly among the groups at any point throughout the study. Mean weekly food intake per mouse was significantly higher in both treatment groups (26.8±3.9 g for flax and 25.8±3.2 g for fish group) as compared to control (22.6±2.8 g). Mean food intake per mouse over the entire experimental course was 18% and 23% higher in the flax and fish groups, respectively, as compared to control. However, this apparent higher food intake was not associated with higher body weight in the treated groups as compared to controls.

### Plasma Lipids

All the animals had comparable plasma lipid levels at baseline. As shown in Table 2, no significant differences in plasma TAG concentrations were observed among the three groups of mice throughout the experimental course. Repeated measures analysis revealed a significant (P<0.001) effect of time. Compared to baseline data, all groups of mice had a significantly lower level of TAG at week four and the end of the study. Compared to the baseline data, the reductions in plasma TAG levels after 4 weeks were 55%, 62%, and 60% in the control, flax, and fish group, respectively.

At baseline, plasma TC concentrations were comparable among groups. However consumption of the supplemented high fat diets resulted in significant increases in plasma TC levels in all of experimental groups by week 4 of the study. Compared to baseline data, the increases in plasma TC levels were 84%, 35% and 22% in the control, fish- and flax-treated animals, respectively. This indicates that the extent of increases in plasma TC levels was significantly affected by the experimental diets. Similar differences in the TC levels among the experimental groups were also observed at week 16. At week 16, mean HDL-cholesterol concentrations in the fish group were 27% and 78% higher than those in either control or flax group, respectively. Plasma lipid data are summarized in Table 2.

### Liver Lipid and Fatty Acid Composition

Total liver lipid content was significantly lower in the fish and flax groups as compared to controls (Table 3); the extent of the reduction in lipid levels in the liver from the fish group was almost twice that in the liver samples from the flax group (−56% vs −24%; p<0.05). No significant differences were observed in the levels of liver TC, TAG, or totals of other lipids, which include phospholipids, when expressed as a percent of total tissue lipid among the groups (Table 3).

The effect of dietary treatment on liver fatty acid composition was studied in total phospholipids (PL), individual phospholipids [phosphatidylethanolamine (PE), phosphatidylcholine (PC), lysophosphatidylcholine (lysoPC), phosphatidylinositol (PI), sphingomyelin (SM) and phosphatidylserine (PS)], free fatty acids (FFA), TAG and cholesteryi esters (CE). Both treatment groups, flax and fish, displayed significantly lower n-6:n-3 ratios in these lipid fractions (except SM fraction) compared to control. However, the flax group had significantly higher ratios in total and individual PL fractions (PE, PC, lysoPC, and PI), as compared to those in the fish group. These ratios did not differ between the flax and fish groups in TAG, FFA, and CE fractions.

The levels of EPA and DHA in various lipid fractions, including individual PL, were significantly higher in the fish group as compared to the flax and control groups. Compared to the control group, the flax group had higher levels of DHA in FFA fraction, higher levels of EPA in PE fraction and higher levels of both EPA and DHA in TAG, PC, PI and PS fractions. Furthermore, consumption of both ‘designer oils’ was comparably associated with a significant reduction in ARA levels in almost all of the lipid fractions tested as compared to the controls. Fatty acid composition and n-6:n-3 FA ratio of liver PL, TAG, FFA, and CE are listed in Table 4, and individual phospholipids of liver in Table 5.

### Materials and Methods

### Animals and Diets

Twenty-one, six week old male C57BL/6 wild type mice were obtained from Central Animal Facility (Winnipeg, Manitoba, Canada). They were given three weeks to acclimate to our facilities with free access to standard mouse chow and water. The mice were maintained in a temperature-controlled room, with a 12:12-hour light-dark cycle. The mice were randomly divided into three groups (n=7) receiving three different diets for 16 weeks. Three different oil formulations were made using flaxseed oil, fish oil, safflower oil, and beef tallow to generate “designer oils” with high and low n-6:n-3 fatty acid ratios and similar background fatty acid composition. The PicoLab mouse diet was supplemented with 2% cholesterol (w/w) plus 0.2% (w/w) cholic acid (base diet). The “control group” received the “base diet” supplemented with a safflower-based oil formulation high in n-6:n-3 fatty acid ratio (25:1). The “flax group” or “fish group” received the “base diet” supplemented with a flaxseed or fish oil-based oil formulation, respectively, low in n-6:n-3 fatty acid ratio (approximately 2:1). Fish oil (EPAX 5500 TG) was a generous gift from EPAX AS, Lysaker, Norway, while flaxseed oil, safflower oil and beef tallow were purchased from DYETS Inc; Bethlehem, Pa. Final composition of the experimental diets is summarized in Table 1. This is a new and well-controlled approach for investigation of low n-6:n-3 FAs in a high fat diet condition. Furthermore, safflower oil was used primarily as the “control oil” due to the lack of n-3 FAs. Body weight and food intake were measured regularly. During the study period, 2 mice from the fish group and 2 mice from the control group were euthanized due to weight loss, dehydration, and signs of jaundice. At sacrifice the remaining mice appeared to be free of these conditions. We speculate that the cholic acid contents of the diet may have contributed to formation of gallstones and other symptoms. At the end of the study, the remaining animals were sacrificed using CO2 gas; autopsy inspection was performed and final blood samples were taken from the heart. Liver tissues were collected and stored at −80° C. until analysis. This study was approved by the Animal Care Committee on the use of animals in Research at the University of Manitoba, Winnipeg, Manitoba, Canada.

### Plasma Lipids

Plasma samples were prepared at baseline, week 4 and the end of the study and used for estimation of total cholesterol (TC), triacylglycerol (TAG), and HDL-cholesterol concentrations using standard enzymatic assays as previously described [31].

### Tissue Lipid Analysis

Lipid extraction of liver tissues was carried out according to Folch et al. [15]. The tissue was weighed prior to extraction and lipid was measured following extraction to determine percentage lipid of total liver weight. Thin-layer chromatography with G-silica gel and H-silica gel was used to separate neutral lipids and phospholipids, respectively, as previously described [38]. Fatty acid analysis was conducted by gas chromatography as described by Park et al. [35]. The levels of TC and TAG in the heart and liver tissues were analyzed using standard enzymatic assays according to manufacture's instructions (Diagnostic Chemicals Limited, Charlottetown, PEI, Canada).

### Statistical Analysis

Data were analyzed using one-way ANOVA followed by the Tukey test for determination of significant differences among the groups using SPSS for Windows version 11.5 (SPSS Inc, Chicago, Ill.). To detect the effects of interaction between time and diets on plasma lipids, Repeated measures analysis was performed. Data are expressed as mean ±standard deviation. Differences among the groups are considered significant at P<0.05.

While the preferred embodiments of the invention have been described above, it will be recognized and understood that various modifications may be made therein, and the appended claims are intended to cover all such modifications which may fall within the spirit and scope of the invention.

### REFERENCES

### Literature Cited

1. Abedin L, Lien E L, Vingrys A J, Sinclair A J (1999) The effects of dietary alpha-linolenic acid compared with docosahexaenoic acid on brain, retina, liver, and heart in the guinea pig. Lipids 34:475-482
2. Alwayn I P, Andersson C, Zauscher B, Gura K, Nose V, Puder M (2005) Omega-3 fatty acids improve hepatic steatosis in a murine model: potential implications for the marginal steatotic liver donor. Transplantation 79:606-608
3. Ander B P, Weber A R, Rampersad P P, Gilchrist J S, Pierce G N, Lukas A (2004) Dietary flaxseed protects against ventricular fibrillation induced by ischemia-reperfusion in normal and hypercholesterolemic rabbits. J Nutr 134:3250-3256
4. Barcelli U O, Beach D C, Pollak V E (1988) The influence of n-6 and n-3 fatty acids on kidney phospholipids composition and on eicosanoid production in aging rats. Lipids 23:309-312
5. Barcelo-Coblijn G, Collison L W, Jolly C A, Murphy E J (2005) Dietary α-linolenic acid increases brain but not heart and liver docosahexaenoic acid levels. Lipids 40:787-798
6. Brenner RR (1974) The oxidative desaturation of unsaturated fatty acids in animals. Mol Cell Biochem 3:41-52
7. Calabresi L, Villa N, Canavesi M, Sirtori C R, James R W, Bernini F, et al (2004) An omega-3 polyunsaturated fatty acid concentrate increases plasma high-density lipoprotein 2 cholesterol and paraoxonase levels in patients with familial combined hyperlipidemia. Metabolism 53:153-158
8. Chrysohoou C, Panagiotakos D B, Pitsavos C, Skoumas J, Krinos X, Chloptsios Y, Nikolaou V, Stefanadis C (2007) Long-term fish consumption is associated with protection against arrhythmia in healthy persons in a Mediterranean region—the ATTICA study. Am J Clin Nutr 85:1385-1391
9. Du C, Sato A, Watanabe S, Wu C, Ikemoto A, Ando K, Kikugawa K, Fujii Y, Okuyama H (2003) Cholesterol synthesis in mice is suppressed but lipofuscin is not affected by long-term feeding of n-3 fatty acid-enriched oils compared with lard and n-6 fatty acid-enriched oils. Biol Pharm Bull 26:766-770
10. Culp B R, Lands W E, Lucches B R, Pitt B, Romson J (1980) The effect of dietary supplementation of fish oil on experimental myocardial infarction. Prostaglandins 20:1021-1031
11. Davidson M H (2006) Mechanisms for the hypotriglyceridemic effect of marine omega-3 fatty acids. Am J Cardiol 98:27i-33i
12. Djousse L, Pankow J S, Eckfeldt J H, Folsom A R, Hopkins P N, Province M A, Hong Y, Ellison R C (2001) Relation between dietary linolenic acid and coronary artery disease in the National Heart, Lung, and Blood Institute Family Heart Study. Am J Clin Nutr 74:612-619
13. Dupasquier C M, Weber A M, Ander B P, Rampersad P P, Steigerwald S, Wigle J T, Mitchell R W, Kroeger E A, Gilchrest J S, Moghadasian M M, Lukas A, Pierce G N (2006) Effects of dietary flaxseed on vascular contractile function and atherosclerosis during prolonged hypercholesterolemia in rabbits. Am J Physiol Heart Circ Physiol 291:H2987-H2996
14. Endres S, Ghorbani R, Kelley V E, Georgilis K, Lonnemann G, van der Meer J W, Cannon J G, Rogers T S, Klempner M S, Weber P C, et al (1989) The effect of dietary supplementation with n-3 polyunsaturated fatty acids on the synthesis of interleukin-1 and tumor necrosis factor by mononuclear cells. N Engl J Med 320:265-271
15. Folch J, Les M, Sloane-Stanley G H (1957) A simple method for the isolation and purification of total lipids from animals. J Biol Chem 226:497-509
16. Fritsche K L, Byrge M, Feng C (1999) Dietary omega-3 polyunsaturated fatty acids from fish oil reduce interleukin-12 and interferon-gamma production in mice. Immunol Lett 65:167-173
17. Gallai V, Sarchielli P, Trequattrini A, Franceschini M, Floridi A, Firenzi C, Alberti A, Di Benedetto D, Stragliotto E (1995) Cytokine secretion and eicosanoid production in the peripheral blood mononuclear cells of MS patients undergoing dietary supplementation with n-3 polyunsaturated fatty acids. J Neuroimmunol 56:143-153
18. Geleijnse J M, Giltay E J, Grobbee D E, Donders A R, Kok F J (2002). Blood pressure response to fish oil supplementation: meta-regression analysis of randomized trial. J Hypertens 20:1493-1499
19. Gerster H (1998) Can adults adequately convert alpha-linolenic acid (18:3n-3) to eicosapentaenoic acid (20:5n-3) and docosahexaenoic acid (22:6n-3)? Int J Vitam Nutr Res 68:159-173
20. Goyens P L, Mensink R P (2005) The dietary alpha-linolenic acid to linoleic acid ratio does not affect the serum lipoprotein profile in humans. J Nutr 135:2799-804
21. Harper C R, Edwards M J, DeFilipis A P, Jacobson T A (2006) Flaxseed oil increases the plasma concentrations of cardioprotective (n-3) fatty acids in humans. J Nutr 136:83-87
22. Harris W S, Bulchandani D (2006) Why do omega-3 fatty acids lower serum triglycerides? Curr Opin Lipidol 17:387-393
23. Hu F B, Bronner L, Willett W C, Stampfer M J, Rexrode K M, Albert C M, Hunter D, Manson J E (2002) Fish and omega-3 fatty acid intake and risk of coronary heart disease in women. JAMA 287:1815-1821
24. Ishida B Y, Blanche P J, Nichols A V, Yasher M, Paigen B (1991) Effects of atherogenic diet consumption on lipoproteins in mouse strains C57BL/6 and C3H. J Lipid Res 32:559-568
25. James M J, Gibson R A, Cleland L G (2000) Dietary polyunsaturated fatty acids and inflammatory mediator production. Am J Clin Nutr 71 (suppl):343S-348 S
26. Kudo N, Kawashima Y (1997) Fish oil-feeding prevents perfluorooctanoic acid-induced fatty liver in mice. Toxicol Appl Pharmacol 145:285-293
27. Maki K C, Van Elswyk M E, McCarthy D, Seeley M A, Veith P E, Hess S P, Ingram K A, Halvorson J J, Calaguas E M, Davidson M H (2003) Lipid responses in mildly hypertriglyceridemic men and women to consumption of docosahexaenoic acid-enriched eggs. Int J Vitam Nutr Res 73:357-368
28. McLennan P L (1993) Relative effects of dietary saturated, monounsaturated, and polyunsaturated fatty acids on cardiac arrhythmias in rats. Am J Clin Nutr 57:207-212
29. McLennan P L, Abeywardena M Y, Charnock J S (1988) Dietary fish oil prevents ventricular fibrillation following coronary artery occlusion and reperfusion. Am Heart J 116:709-717
30. McLennan P L, Bridle T M, Abeywardena M Y, Charnock J S (1993) Comparative efficacy of n-3 and n-6 polyunsaturated fatty acids in modulating ventricular fibrillation threshold in marmoset monkeys. Am J Clin Nutr 58:666-669
31. Moghadasian M H, McManus B M, Pritchard P H, Frohlich J J (1997) “Tall oil”-derived phytosterols reduce atherosclerosis in ApoE-deficient mice. Arterioscler Thromb Vasc Biol 17:119-126
32. Mori T A, Burke V, Puddey I B, Watts G F, O'Neal D N, Best J D, Beilin L J (2000) Purified eicosapentaenoic and docosahexaenoic acids have differential effects on serum lipids and lipoproteins, LDL particle size, glucose, and insulin in mildly hyperlipidemic men. Am J Clin Nutr 71:1085-1094
33. Neschen S, Morino K, Rossbacher J C, Pongratz R L, Cline G W, Sono S, Gillum M, Shulman G I (2006) Fish oil regulates adiponectin secretion by a peroxisome proliferators-activated receptor-gamma-dependent mechanism in mice. Diabetes 55:924-928
34. Owen A J, Peter-Przyborowska B A, Hoy A J, McLennan P L (2004) Dietary fish oil dose- and time-response effects on cardiac phospholipid fatty acid composition. Lipids 39:955-961
35. Park E J, Suh M, Ramanujam K, Steiner K, Begg D, Clandinin M T (2005) Diet-induced changes in membrane gangliosides in rat intestinal mucosa, plasma and brain. J Pediatr Gastroenterol Nutr 40:487-495
36. Simopoulos A P (2002) The importance of the ratio of omega-6/omega-3 essential fatty acids. Biomed Pharmacother 56:365-379
37. Simopoulos AP (1999) Essential fatty acids in health and chronic disease. Am J Clin Nutr 70(suppl):560S-569S
38. Suh M, Wierzbicki A A, Clandinin M T (1994) Dietary fat alters membrane composition in rod outer segments in normal and diabetic rats: impact on content of very-long-chain (C≧24) polyenoic fatty acids. Biochim Biophys Acta 1214:54-62
39. Wang S, Wu D, Matthan N, Lichtenstein A (2007) The impact of different ratios of omega-6 polyunsaturated fatty acids to eicosapentaenoic acid (EPA) plus docosahexaenoic acid (DHA) on atherosclerotic lesion formation and inflammatory factors in the LDL receptor knockout (LDLr-/-) mouse. FASEB J 21:231.1
40. Xu Z, Riediger N, Innis S, Moghadasian M H (2007) Fish oil significantly alters fatty acid profiles in various lipid fractions but not atherogenesis in apo E-KO mice. Eur J Nutr Epub ahead of print
41. Yamashita T, Oda E, Sano T, Yamashita T, Ijiru Y, Giddings J C, Yamamoto J (2005) Varying the ratio of dietary n-6:n-3 polyunsaturated fatty acid alters the tendency to thrombosis and progress of atherosclerosis in apoE-/- LDLR-/- double knockout mouse. Thromb Res 116:393-401
42. Yamazaki T, Nakamori A, Sasaki E, Wada S, Ezaki 0 (2007) Fish oil prevents sucrose-induced fatty liver but exacerbates high-safflower oil-induced fatty liver in ddy mice. Hepatology 46:1779-1790.
43. Zhao G, Etherton T D, Martin K R, West S G, Gillies P J, Kris-Etherton PM (2004) Dietary alpha-linolenic acid reduces inflammatory and lipid cardiovascular risk factors in hypercholesterolemic men and women. J Nutr 134:2991-2997

TABLE 1
Composition of experimental diets per 100 g

| | Experimental Diets | | |
|---|---|---|---|
| | Control diet | Flax diet | Fish diet |
| Mouse chow¹ | 87.8 | 87.8 | 87.8 |
| Cholesterol | 2.0 | 2.0 | 2.0 |
| Cholic acid | 0.2 | 0.2 | 0.2 |
| Designer oil² | 10.0 | 10.0 | 10.0 |
| Fatty acid³ (%, w/w) | | | |
| 16:0 | 12.4 | 12.5 | 12.7 |
| 18:0 | 5.6 | 6.4 | 6.7 |
| 18:1 (7 & 9) | 20.7 | 23.7 | 21.7 |
| 18:2 n-6 | 53.7 | 35.1 | 35.7 |
| 18:3 n-3 | 1.5 | 15.9 | 1.9 |
| 20:5 n-3 | 0.3 | 0.3 | 7.0 |
| 22:6 n-3 | 0.2 | 0.3 | 4.6 |
| n-6:n-3 ratio | 25.9 | 2.2 | 2.6 |
| ΣSaturated | 19.6 | 20.4 | 21.1 |
| ΣMonounsaturated | 22.7 | 26.0 | 25.2 |
| ΣPolyunsaturated | 56.1 | 52.0 | 51.2 |

¹Pico Lab mouse diet containing (g/100 g unless noted) protein (20.5), fat (9), cholesterol (285 mg/kg), carbohydrate (53), ash and vitamins (4.8), fiber (2.7), moisture (10) was used to prepare experimental diets
²Each group had different mixture of oils (safflower, fish, flaxseed) and beef tallow to obtain desired ratio
³Presented as % w/w of total diet lipid

TABLE 2
Plasma lipid levels (triglyceride, total cholesterol, HDL-, and
non-HDL-cholesterol) throughout the study course from the
three groups of experimental mice.

| | Control | Flax | Fish |
|---|---|---|---|
| Plasma lipids | (n = 5-7) | (n = 7) | (n = 5-7) |
| Triglycerides (mmol/L) | | | |
| Week 0 | 1.05 ± 0.38 | 0.90 ± 0.21 | 0.94 ± 0.19 |
| Week 4¹ | 0.47 ± 0.13 | 0.34 ± 0.07 | 0.38 ± 0.07 |
| Week 16¹ | 0.50 ± 0.14 | 0.52 ± 0.08 | 0.43 ± 0.11 |
| Total cholesterol (mmol/L) | | | |
| Week 0 | 1.72 ± 0.76 | 1.56 ± 0.34 | 1.50 ± 0.20 |
| Week 4¹ | 3.18 ± 0.86^{a} | 2.11 ± 0.23^{b} | 1.83 ± 0.13^{b} |
| Week 16¹ | 3.31 ± 0.40^{a} | 1.12 ± 0.63^{b} | 2.41 ± 0.39^{b} |
| HDL-C (mmol/L)² | | | |
| Week 4 | 1.45 ± 0.81 | 0.85 ± 0.15 | 0.96 ± 0.16 |
| Week 16 | 1.33 ± 0.18^{a} | 0.94 ± 0.06^{a} | 1.69 ± 0.56^{b} |
| Non-HDL-C (mmol/L)² | | | |
| Week 4 | 1.73 ± 0.24^{a} | 1.26 ± 0.22^{b} | 1.06 ± 0.47^{b} |
| Week 16 | 1.98 ± 0.38^{a} | 1.18 ± 0.59^{b} | 0.98 ± 0.24^{b} |

Values are mean +/− SD; values with different superscript letters within a row are significantly different.
¹Significant (P < 0.001) effect of time on respective plasma lipid as compared to baseline data
²Insufficient plasma samples at weeks 0 for HDL-cholesterol measurements

TABLE 3
Liver lipid compositions of experimental animals

| | Control | Flax | Fish |
|---|---|---|---|
| Total liver lipid | 22.06 ± 3.17^{a} | 16.79 ± 3.68^{b} | 9.81 ± 2.32^{c} |
| (% of total tissue | | | |
| weight) | | | |
| Total cholesterol | 15.99 ± 4.58 | 15.12 ± 4.63 | 11.09 ± 7.17 |
| (% of total liver lipid) | | | |
| Total triglycerides | 6.45 ± 1.29 | 6.50 ± 0.94 | 8.14 ± 2.38 |
| (% of total liver lipid) | | | |
| Other lipids | 77.57 ± 5.40 | 78.38 ± 5.12 | 80.76 ± 8.55 |
| (% of total liver lipid)¹ | | | |

Values are mean +/− SD; values with different superscript letters within a row are significantly different.
¹Other lipids includes phospholipids, free fatty acids, diglycerides, monoglycerides and cholesteryl esters

TABLE 4
Fatty acid composition of liver lipid fractions of the three groups of experimental mice.

| | Phospholipids | | | Triglycerides | | |
|---|---|---|---|---|---|---|
| Fatty acid | Control | Flax | Fish | Control | Flax | Fish |
| 16:0 | 26.6 ± 1.1 | 25.5 ± 3.7 | 23.1 ± 1.0 | 7.8 ± 1.2 | 8.5 ± 2.4 | 5.8 ± 1.3 |
| 16:1 (5 & 7) | 2.0 ± 0.2^{a} | 2.0 ± 0.3^{a} | 1.5 ± 0.4^{b} | 3.3 ± 1.1 | 3.4 ± 0.8 | 2.1 ± 0.7 |
| 18:0 | 14.1 ± 1.4 | 14.2 ± 2.6 | 16.2 ± 2.2 | 1.6 ± 0.3 | 2.7 ± 2.3 | 2.2 ± 0.6 |
| 18:1 (7 & 9) | 19.0 ± 1.4 | 19.3 ± 1.6 | 17.0 ± 1.9 | 38.4 ± 2.4^{ab} | 41.3 ± 2.1^{a} | 35.1 ± 2.0^{b} |
| 18:2 | 19.9 ± 1.1 | 18.5 ± 3.1 | 17.5 ± 0.7 | 37.0 ± 1.3^{a} | 26.5 ± 2.2^{b} | 26.9 ± 1.6^{b} |
| 18:3 (3) | ND | 0.7 ± 0.3^{b} | 0.1 ± 0.1^{a} | 0.2 ± 0.2^{a} | 5.0 ± 0.9^{b} | 0.7 ± 0.1^{a} |
| 20:0 | 0.5 ± 0.1 | 0.7 ± 0.9 | 0.6 ± 0.1 | 0.4 ± 0.3^{a} | 0.3 ± 0.2^{b} | 0.7 ± 0.3^{a} |
| 20:1 | 0.6 ± 0.1 | 0.6 ± 0.1 | 0.7 ± 0.1 | 2.2 ± 0.4 | 2.1 ± 0.4 | 2.3 ± 0.3 |
| 20:2 (6) | 0.7 ± 0.1^{a} | 0.5 ± 0.1^{b} | 0.5 ± 0.2^{b} | 1.7 ± 0.4 | 1.2 ± 1.3 | 0.5 ± 0.1 |
| 20:3 (6) | 2.6 ± 0.2^{a} | 2.4 ± 0.6^{a} | 1.2 ± 0.3^{b} | 3.5 ± 0.3^{a} | 1.5 ± 0.4^{b} | 0.6 ± 0.2^{c} |
| 20:4 (6) | 7.0 ± 1.4^{a} | 4.6 ± 1.5^{b} | 3.9 ± 0.3^{b} | 1.2 ± 0.2^{a} | 0.6 ± 0.5^{b} | 0.5 ± 0.1^{b} |
| 20:5 (3) | 0.2 ± 0.3^{a} | 1.4 ± 0.6^{b} | 3.5 ± 0.6^{c} | 0 ± 0.1^{a} | 0.5 ± 0.2^{b} | 2.4+ ± 0.2^{c} |
| 22:5 (3) | ND | 0.7 ± 0.2^{b} | 1.1 ± 0.1^{c} | 0.2 ± 0.2^{a} | 1.1 ± 0.8^{a} | 4.1 ± 0.9^{b} |
| 22:6 (3) | 3.4 ± 0.5^{a} | 6.0 ± 2.0^{b} | 10.0 ± 1.0^{c} | 1.8 ± 0.1^{a} | 4.4 ± 1.1^{b} | 15.0 ± 2.5^{c} |
| n-6:n-3 ratio | 8.4 ± 0.4^{a} | 3.1 ± 0.5^{b} | 1.6 ± 0.1^{c} | 21.3 ± 5.3^{a} | 2.7 ± 0.2^{b} | 1.3 ± 0.2^{b} |
| | Free fatty acids | | | Cholesteryl esters | | |
| Fatty acid | Control | Flax | Fish | Control | Flax | Fish |
| 16:0 | 15.0 ± 1.2^{a} | 14.8 ± 0.6^{a} | 17.5 ± 2.3^{b} | 5.3 ± 0.4^{a} | 6.2 ± 1.0^{a} | 11.3 ± 5.7^{b} |
| 16:1 (5 & 7) | 3.6 ± 1.1 | 4.0 ± 0.6 | 2.9 ± 0.8 | 11.3 ± 1.3^{ab} | 12.2 ± 0.8^{a} | 9.2 ± 2.5^{b} |
| 18:0 | 3.0 ± 1.8^{a} | 3.7 ± 0.6^{a} | 5.4 ± 0.7^{b} | 0.5 ± 0.1^{a} | 0.7 ± 0.1^{a} | 1.7 ± 0.9^{b} |
| 18:1 (7 & 9) | 28.2 ± 2.4^{ab} | 30.2 ± 2.5^{a} | 26.2 ± 1.4^{b} | 50.9 ± 0.6 | 53.5 ± 1.9 | 51.5 ± 5.2 |
| 18:2 | 35.7 ± 2.7^{a} | 29.5 ± 2.0^{b} | 28.7 ± 2.4^{b} | 28.7 ± 1.4^{a} | 16.5 ± 1.0^{b} | 19.1 ± 2.1^{c} |
| 18:3 (3) | ND | 4.3 ± 1.1^{b} | 0.5 ± 0.3^{a} | 0.7 ± 0.1^{a} | 8.4 ± 1.7^{b} | 1.5 ± 0.2^{a} |
| 20:0 | ND | ND | 0.232 ± 0.2^{b} | 0.2 ± 0.01 | 0.2 ± 0.01 | 0.2 ± 0.2 |
| 20:1 | 1.0 ± 0.2 | 1.0 ± 0.2 | 0.9 ± 0.1 | ND | ND | ND |
| 20:2 (6) | 1.4 ± 0.4^{a} | 0.9 ± 0.5^{ab} | 0.3 ± 0.2^{b} | ND | ND | ND |
| 20:3 (6) | 2.9 ± 0.1^{a} | 1.8 ± 0.3^{b} | 0.8 ± 0.3^{c} | 0.4 ± 0.1^{a} | 0.1 ± 0.1^{b} | 0.1 ± 0.1^{b} |
| 20:4 (6) | 5.7 ± 0.5^{a} | 3.7 ± 0.8^{b} | 2.9 ± 0.4^{b} | 0.4 ± 0.1^{a} | 0.1 ± 0.1^{b} | 0.1 ± 0.2^{b} |
| 20:5 (3) | 1.5 ± 1.7^{a} | 1.6 ± 0.5^{a} | 4.7 ± 0.7^{b} | ND | 0.2 ± 0.1^{a} | 1.0 ± 0.3^{b} |
| 22:5 (3) | ND | 0.4 ± 0.5^{ab} | 1.2 ± 1.1^{b} | ND | ND | 0.2 ± 0.2^{b} |
| 22:6 (3) | 2.1 ± 0.4^{a} | 3.7 ± 0.7^{b} | 7.2 ± 1.2^{c} | 0.3 ± 0.1^{a} | 0.5 ± 0.1^{a} | 2.6 ± 1.0^{b} |
| n-6:n-3 ratio | 16.6 ± 8.6^{a} | 3.7 ± 0.9^{b} | 2.5 ± 0.5^{b} | 27.9 ± 3.2^{a} | 1.9 ± 0.3^{b} | 3.8 ± 0.6^{b} |

Values are presented as %(w/w) with mean +/− SD (n = 5-7).
Values with different superscript letter in a row within a lipid fraction are significantly different, p < 0.05;
ND, Not Detected

TABLE 5
Selected fatty acid profiles in the individual phospholipid fractions
of the liver tissues from the three groups of mice.

| Phospholipid fraction and | Fatty acid as a proportion of total fatty acids (% w/w) | | | | | |
|---|---|---|---|---|---|---|
| group | 18:3 (n-3) | 20:4 (n-6) | 20:5 (n-3) | 22:5 (n-3) | 22:6 (n-3) | n-6/n-3 ratio |
| Phosphatidylethanolamine | | | | | | |
| Control | ND | 16.3 ± 2.7^{a} | 0.3 ± 0.1^{a} | 0.5 ± 0.1^{a} | 11.1 ± 1.8^{a} | 2.7 ± 0.3^{a} |
| Flax | 1.4 ± 0.6 | 8.6 ± 1.8^{b} | 2.4 ± 0.7^{b} | 1.3 ± 0.2^{b} | 13.2 ± 2.5^{a} | 1.4 ± 0.3^{b} |
| Fish | ND | 5.1 ± 0.5^{b} | 5.3 ± 0.5^{c} | 1.8 ± 0.4^{c} | 21.1 ± 1.0^{b} | 0.5 ± 0.0^{c} |
| Phosphatidylcholine | | | | | | |
| Control | 0.0 ± 0.1^{a} | 9.0 ± 1.5^{a} | 0.2 ± 0.04^{a} | 0.2 ± 0.0^{a} | 5.4 ± 0.6^{a} | 7.0 ± 0.7^{a} |
| Flax | 1.1 ± 0.3^{b} | 4.4 ± 0.7^{b} | 1.7 ± 0.3^{b} | 0.6 ± 0.1^{b} | 7.6 ± 1.3^{b} | 3.0 ± 0.5^{b} |
| Fish | 0.2 ± 0.0^{a} | 3.9 ± 0.4^{b} | 5.0 ± 1.0^{c} | 1.3 ± 0.2^{c} | 12.5 ± 0.7^{c} | 1.5 ± 0.2^{c} |
| Phosphatidylserine | | | | | | |
| Control | ND | 8.7 ± 3.4^{a} | ND | 1.1 ± 0.3^{a} | 9.0 ± 3.2^{a} | 2.0 ± 0.43^{a} |
| Flax | ND | 6.1 ± 1.2^{ab} | ND | 1.6 ± 0.2^{b} | 14.8 ± 2.7^{b} | 1.1 ± 0.4^{b} |
| Fish | ND | 3.5 ± 0.2^{b} | ND | 2.3 ± 0.4^{c} | 24.1 ± 1.4^{c} | 0.6 ± 0.5^{b} |
| Phosphatidylinositol | | | | | | |
| Control | 0.2 ± 0.1 | 21.9 ± 1.9^{a} | 0.1 ± 0.2^{a} | 0.2 ± 0.0^{a} | 2.1 ± 0.2^{a} | 17.2 ± 1.5^{a} |
| Flax | 0.1 ± 0.1 | 16.2 ± 1.3^{b} | 1.5 ± 0.3^{b} | 0.9 ± 0.1^{b} | 3.6 ± 0.8^{b} | 6.0 ± 0.9^{b} |
| Fish | 0.2 ± 0.1 | 14.4 ± 1.3^{b} | 4.9 ± 0.9^{c} | 2.4 ± 0.4^{c} | 8.4 ± 0.7^{c} | 1.9 ± 0.2^{c} |
| Lysophosphatidylcholine | | | | | | |
| Control | 0.1 ± 0.1 | 5.6 ± 1.3^{a} | 0.0 ± 0.1^{a} | 0.3 ± 0.2^{a} | 5.0 ± 0.7^{a} | 5.5 ± 0.4^{a} |
| Flax | 0.0 ± 0.1 | 2.9 ± 0.8^{b} | 1.2 ± 0.1^{b} | 0.5 ± 0.0^{a} | 6.6 ± 1.2^{a} | 2.8 ± 0.6^{b} |
| Fish | ND | 2.2 ± 0.3^{b} | 3.3 ± 0.4^{ab} | 1.4 ± 0.2^{b} | 10.8 ± 1.1^{b} | 1.2 ± 0.2^{c} |
| Sphingomyelin | | | | | | |
| Control | 0.2 ± 0.2 | 1.0 ± 0.2^{a} | 0.1 ± 0.2^{a} | ND | 1.4 ± 0.2^{a} | 8.2 ± 3.2 |
| Flax | 0.2 ± 0.2 | 0.4 ± 0.2^{b} | ND^{a} | ND | 1.4 ± 0.5^{a} | 6.7 ± 2.7 |
| Fish | 0.0 ± 0.1 | 0.4 ± 0.1^{b} | 0.4 ± 0.1^{b} | ND | 2.0 ± 0.6^{a} | 4.9 ± 0.8 |

Values are mean +/− SD (n = 4-7, except for Lysophosphatidylcholine in the fish group in which only 3 samples were analyzed due to contamination of other samples).
Values with different superscript letter for a fatty acid in a phospholipids fraction are significantly different, p < 0.05;
ND, Not Detected

1. An edible oil having an n-6:n-3 ratio of approximately 1.5:1 to 4:1.

2. The edible oil according to claim 1 comprising an n-6:n-3 ratio of approximately 2:1 to 3:1.

3. The edible oil according to claim 1 wherein the designer oil has a fatty acid profile of approximately 30-40% n-6 fatty acid and 10-25% n-3 fatty acid.

4. The edible oil according to claim 1 wherein the designer oil has a fatty acid profile of approximately 33-38% n-6 and 15-20% n-3.

5. A method of improving over-all health comprising administering to an individual in need of such treatment an effective amount of an edible oil having an n-6:n-3 ratio of approximately 1.5:1 to 4:1.

6. The method according to claim 5 comprising an n-6:n-3 ratio of approximately 2:1 to 3:1.

7. The method according to claim 5 wherein the designer oil has a fatty acid profile of approximately 30-40% n-6 fatty acid and 10-25% n-3 fatty acid.

8. The method according to claim 5 wherein the designer oil has a fatty acid profile of approximately 33-38% n-6 and 15-20% n-3.
